(19) United States
(12) Patent Application Publication
(10) Pub. No.: US 2022/0090786 A1
(43) Pub. Date: Mar. 24, 2022

(54) AVERAGING COMBUSTION IN-SITU OXYGEN ANALYZER

(71) Applicant: Rosemount Inc, Shakopee, MN (US)

(72) Inventors: Pavel SHUK, Copley, OH (US); Dirk W. BAUSCHKE, Shakopee, MN (US); David LOBERG, Prior Lake, MN (US); Narendra KULKARNI, Pune (IN); Tejas DUBE, Pune (IN); Tushar SONAWANE, Pune (IN)

(73) Assignee: Rosemount Inc

(21) Appl. No.: 17/234,107

(22) Filed: Apr. 19, 2021

(30) Foreign Application Priority Data
Sep. 24, 2020 (IN) 202021041522

(51) Int. Cl. F23N 5/00; G01N 1/22

(52) U.S. Cl. CPC F23N 5/006; G01N 1/2258

(57) ABSTRACT

An in-situ averaging combustion analyzer includes a housing and a probe coupled to the housing at a proximal end. The probe has a distal end configured to extend into a flue and contains a zirconia-based oxygen sensing cell proximate the distal end. Electronics are disposed in the housing and are coupled to the zirconia-based oxygen sensing cell. The electronics are configured to measure an electrical characteristic of the zirconia-based oxygen sensing cell and calculate an oxygen concentration value. An averaging conduit is disposed about the probe and has a plurality of inlets spaced at different distances from the distal end of the probe. The averaging conduit has at least one outlet positioned between the distal end and the proximal end of the probe. The electronics are configured to provide an average oxygen concentration output based on the calculated oxygen concentration value.

### BACKGROUND

Industrial processes often rely on energy sources such as combustion to generate steam or heat for a feed stock liquid. Some combustion processes involve operation of a furnace or boiler. While combustion provides a relatively low-cost energy source, combustion efficiency is often sought to be maximized within a process, because the resulting flue gases exiting the system may be subject to regulations regarding emissions of harmful gases. Accordingly, one goal of the combustion process management industry is to maximize combustion efficiency of existing furnaces and boilers, which inherently reduces the production of greenhouse gases and other harmful biproducts.

Combustion efficiency can be optimized by maintaining the ideal level of oxygen in the exhaust or flue gases coming from a combustion process, which ensures oxidation of the combustion biproducts. In-situ or in-process analyzers are commonly used in monitoring, optimizing, and/or controlling an on-going combustion process. Typically, such analyzers employ a sensor that is heated to relatively high temperatures and operates directly above or near the furnace or boiler combustion zone.

Known process combustion analyzers typically employ a zirconia-based oxygen sensor disposed at one end of a probe that is inserted into a flue gas stream. As the exhaust/flue gas flows into the sensor, it diffuses through a filter or diffuser into proximity with the zirconia-based oxygen sensor. There are no pumps or other flow inducing devices used to direct sample flow into the sensor. Instead, the gas penetrates passively through the diffuser. The sensor provides an electrical signal related to the amount of oxygen present in the flue gas.

The zirconia-based oxygen sensor provides a potentiometric indication that is deemed a reliable oxygen measurement in combustion environments permitting efficient and safe process control. Typically, a single probe is inserted through a process intrusion or insertion into the exhaust stack. A percent O₂ measurement is used to control combustion efficiency in small boilers. In large boiler installations, operators frequently encounter flue gas stratification with many layers of different oxygen concentrations. In an attempt to obtain stratification information, operators may choose to install multiple (sometimes as many as 16) probes into the exhaust stack for efficient and safe operation.

A typical in-situ analyzer with a zirconia potentiometric oxygen sensor provides a single point oxygen measurement for controlling combustion efficiency in power plants, incinerators, energy saving systems, refineries, chemical plants, or small combustors. As described above, large stacks have considerable flue gas stratification with many different concentration layers in the flue gas. In such cases, it is common in such large combustion applications to utilize multiple oxygen sensing probes. However, the utilization of such probes increases the complexity and expense of the entire combustion control system. For example, each probe requires power/signal wiring, calibration gas lines, and a probe mount fitting.

An alternative for some large combustion applications to provide oxygen stratification information is the utilization of a tunable diode laser oxygen sensor. Such sensors are currently used in applications to provide averaging oxygen concentrations but are generally deemed to 3 or 4 times more costly than a single zirconia oxygen probe and such systems would not have the benefit of periodic in-situ calibration. Further, such tunable diode laser systems rely on laser energy passing through the flue gas and may be limited in instances where the flue gas is partially or completely opaque.

### SUMMARY

An in-situ averaging combustion analyzer includes a housing and a probe coupled to the housing at a proximal end. The probe has a distal end configured to extend into a flue and contains a zirconia-based oxygen sensing cell proximate the distal end. Electronics are disposed in the housing and are coupled to the zirconia-based oxygen sensing cell. The electronics are configured to measure an electrical characteristic of the zirconia-based oxygen sensing cell and calculate an oxygen concentration value. An averaging conduit is disposed about the probe and has a plurality of inlets spaced at different distances from the distal end of the probe. The averaging conduit has at least one outlet positioned between the distal end and the proximal end of the probe. The electronics are configured to provide an average oxygen concentration output based on the calculated oxygen concentration value.

### BRIEF DESCRIPTION OF THE DRAWINGS

FIG. 1 is a diagrammatic view of an in-situ oxygen analyzer/transmitter with which embodiments of the present invention are particularly applicable.

FIG. 2 is a diagrammatic perspective view of a combustion oxygen transmitter with which embodiments of the present invention are particularly applicable.

FIG. 3 is a diagrammatic view illustrating oxygen stratification across a flue duct.

FIG. 4 is a diagrammatic elevation view showing a plurality of single-point oxygen probe/analyzers being used within a flue to provide an average oxygen measurement across the width of flue.

FIG. 5 is a diagrammatic view of an in-situ averaging oxygen sensing probe/analyzer in accordance with an embodiment of the present invention.

FIG. 6 is a diagrammatic view of an averaging oxygen sensing probe/analyzer in accordance with another embodiment of the present invention.

FIGS. 7A and 7B are diagrammatic perspective views of averaging pipes for an in-situ oxygen probe/analyzer in accordance with embodiments of the present invention.

FIGS. 8A-8D show various embodiments of averaging pipes where the diameters of the inlet apertures vary on each averaging pipe.

FIG. 9 is a diagrammatic view of an in-situ oxygen probe/analyzer in accordance with an embodiment of the present invention.

FIG. 10 is a flow diagram of a method of providing an average oxygen concentration of stratified flue gas using a single oxygen probe/analyzer in accordance with an embodiment of the present invention.

### DETAILED DESCRIPTION OF ILLUSTRATIVE EMBODIMENTS

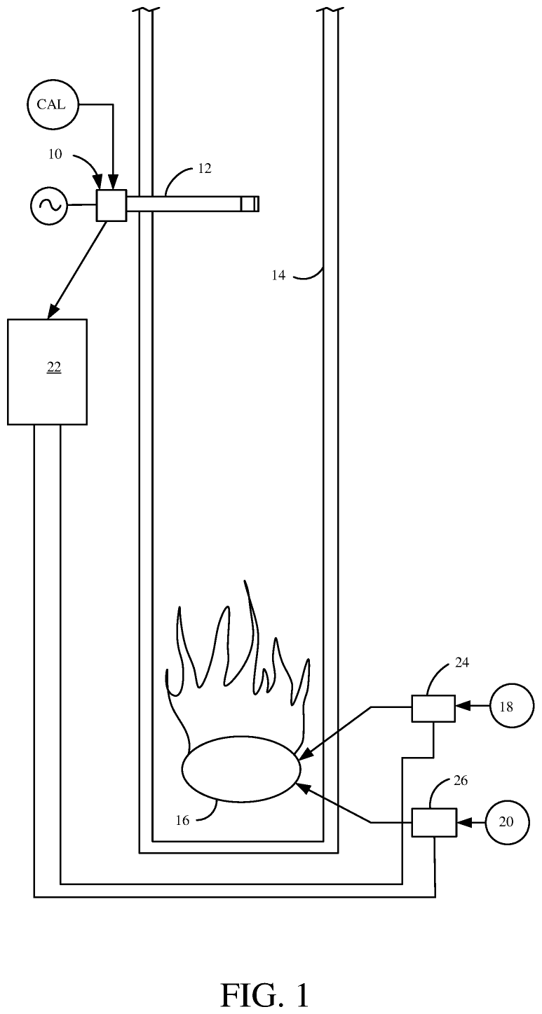

FIG. 1 is a diagrammatic view of an in-situ oxygen analyzer/transmitter with which embodiments of the present invention are particularly applicable. Transmitter 10 may be, for example, a Model 6888 Oxygen Transmitter available from Rosemount Inc. (an Emerson Automation Solutions Company). Transmitter 10 includes a probe assembly 12 substantially disposed within a stack or flue 14 of a combustion process. Transmitter 10 is configured to measure oxygen concentration within the flue gas produced by combustion occurring at burner 16. Burner 16 may be operably coupled to a source of air or other oxygen source 18, as well as a combustion fuel source 20. A combustion controller 22 is operably coupled to oxygen valve 24 and fuel valve 20. Based on signals from combustion controller 22, valves 18 and/or 20 control the air and/or fuel supplied to the combustion process occurring at burner 16. Combustion controller 22 receives an indication of oxygen in the flue gas form transmitter 10 and uses this indication to provide efficient and environmentally friendly control of the combustion process. As transmitter 10 is configured to be exposed to the combustion zone it may be constructed to withstand high temperatures.

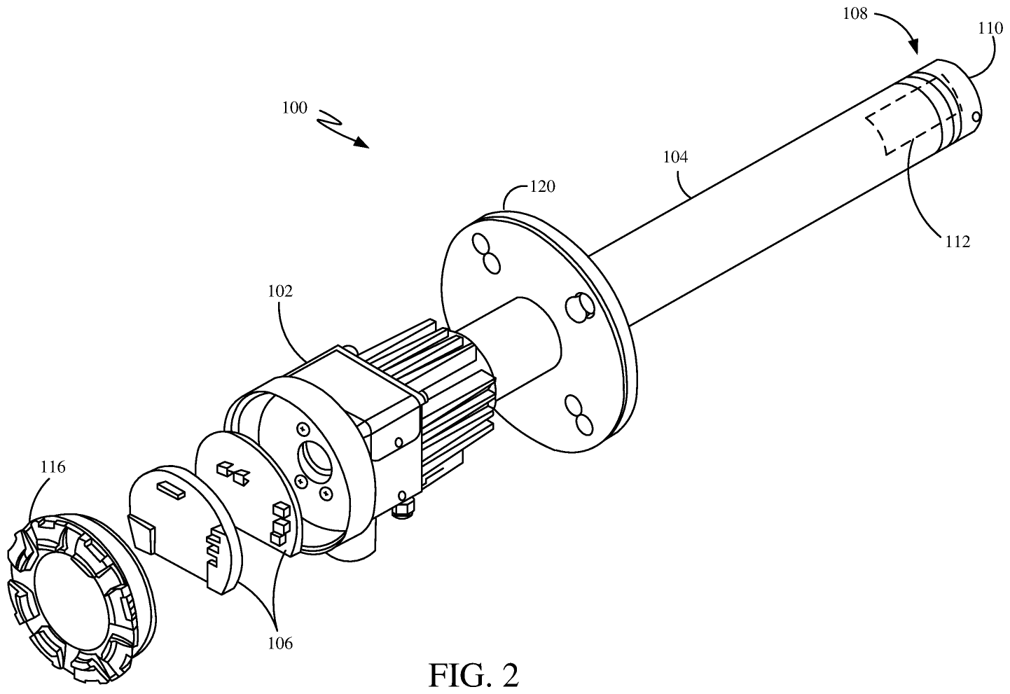

FIG. 2 is a diagrammatic perspective view of a combustion oxygen transmitter with which embodiments of the present invention are particularly applicable. Transmitter 100 includes housing 102, probe 104, and electronics 106. Transmitter 100 is typically mounted to a stack or flue gas wall 104 using flange 120.

Probe 104 includes a distal end 108 where a diffuser or filter 110 is mounted. Diffuser 110 is a physical device that is configured to allow at least some gaseous diffusion therethrough, but otherwise protects components within probe 104. Specifically, diffuser 110 protects a zirconia-based oxygen measurement cell or sensor 112. Zirconia-based oxygen measurement cell 112 utilizes known technology and design to provide a potentiometric or amperometric indication of oxygen in the flue gas when cell 112 is operating within its thermal operating range. Electronics 106 are typically configured to provide thermal control to probe 104 using an electrical heater and temperature sensor (not shown). Additionally, electronics 106 are configured to obtain the amperometric or potentiometric response of cell 112 and calculate an oxygen output. In one example, electronics 106 employs the known Nernst equation for such calculation.

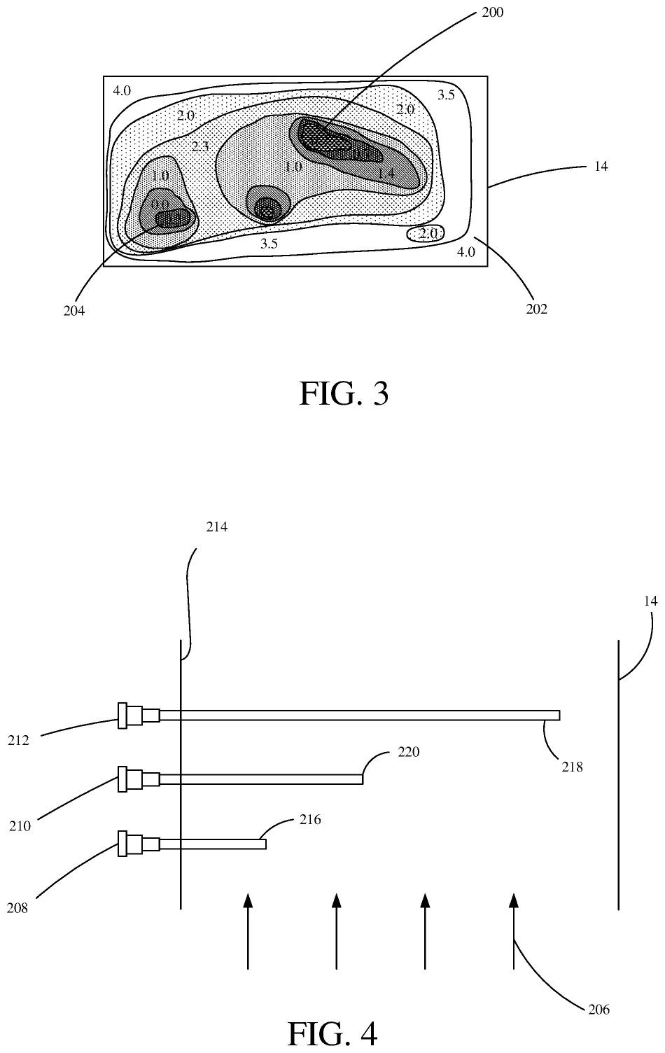

FIG. 3 is a diagrammatic view illustrating oxygen stratification within flue 14. FIG. 3 is a top plan view essentially showing a cross section of the stratification for illustration purposes. As can be seen, the oxygen concentration varies from 0.0% O₂ in region 200 to 4.0% O₂ in region 202. Additionally, a relatively low concentration oxygen island is provided at reference numeral 204 having an oxygen percent of 0.3% O₂. Accordingly, a single probe disposed within the flue will generally measure the oxygen concentration at the location at the distal end of the probe. As can be appreciated, for such a single-point measurement, this may not provide the entire picture with respect to oxygen concentration in the flue when stratification occurs.

FIG. 4 is a diagrammatic elevation view showing a plurality of single-point oxygen probe/analyzers being used within a flue 14 in order to provide an average oxygen measurement across the width of flue 14. As shown, flue gases 206 flow upwardly through flue 14 and stratification will cause different measurements for the different oxygen probe analyzers 208, 210, and 212. This is because the sensing regions of the various oxygen probe/analyzers are disposed at different distances from flue gas wall 214. For example, sensor 216 of oxygen probe/analyzer 208 is disposed relatively close to wall 214. On the other hand, sensor 218 of probe/analyzer 212 is disposed almost all the way across flue 14 and is actually proximate the opposite wall. Further, sensor 220 of oxygen probe/analyzer 210 is positioned near the center of flue 14. Accordingly, if flue gas stratification occurs, the different sensors 216, 218, and 220 will read slightly different oxygen percent readings based on their discrete locations within the stratification. Then, the various oxygen percent values provided by probe/analyzers 208, 210, 212, can be averaged or otherwise combined in order to provide a more accurate indication of flue gas percent oxygen than would be possible with a single probe.

However, as set for the above, the utilization of multiple probe/analyzers to deal with flue gas stratification introduces considerable complexity and expense.

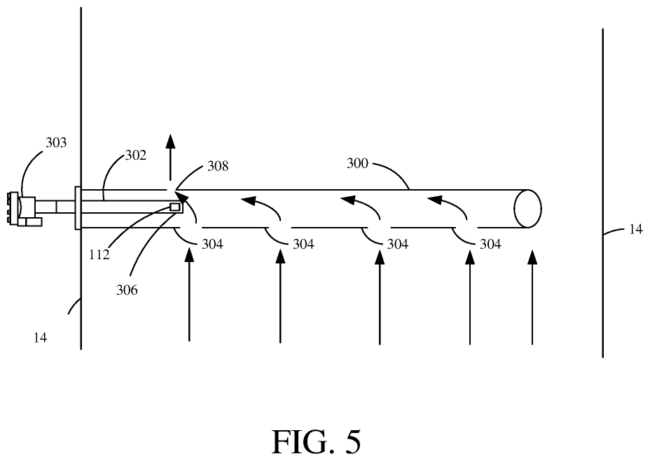

FIG. 5 is a diagrammatic view of an in-situ averaging oxygen sensing probe/analyzers in accordance with an embodiment of the present invention. In many clean natural gas, light oil or even moderate dusty oil/coal combustion applications, an averaging conduit 300 can be installed with a single probe 302 inside. Averaging conduit 300 may mount to a flange of the oxygen transmitter or to the probe 302. Oxygen transmitter 303 includes single probe 302 and can be a legacy or known oxygen transmitter, in one embodiment. Using a single oxygen transmitter 303 with probe 302 and an averaging conduit 300 provides a very cost-effective averaging oxygen measurement.

Conduit 300 has a number of upstream openings 304 that permit flue gas sampling across duct or flue 14. Embodiments provided herein can provide a reliable and cost-effective averaging option compared to the utilization of multiple probes (FIG. 4) or to tunable diode laser-based solutions. Flue gas from different locations across the duct or flue 14 flows through inlets 304 of conduit 300 and is delivered to zirconia-based oxygen sensing cell 112 at distal end 306 of probe 302. Flow through conduit 300 is achieved, in one example, through the suction created by downstream outlet 308, similar to an eductor, with a low and high velocity managed by pipe opening size and Venturi effect. In the embodiment shown in FIG. 5, upstream apertures 304 are positioned facing downwardly as flue gas rises through stack or flue 14. However, embodiments can be practiced where the duct or flue is not vertical. As shown, exit aperture 308 is generally positioned on a downstream side of pipe 300 and is positioned axially (i.e., length along conduit 300) at a same or closer distance from flue wall 14 as sensor 306. In the example shown, exit aperture 308 is actually positioned closer to wall 14 than sensor 306 of probe 302. This ensures that the flue gas is drawn past sensing cell 112 in order to provide the averaging operation.

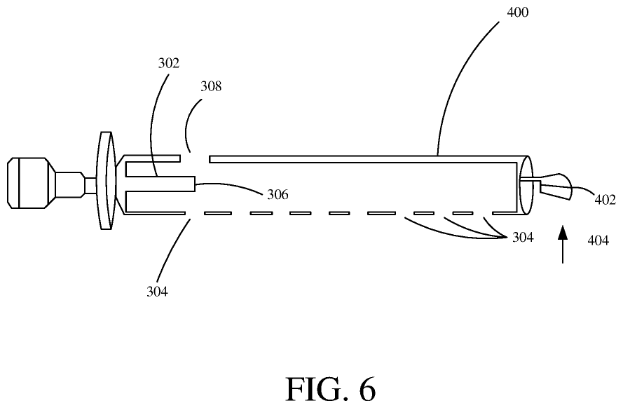

FIG. 6 is a diagrammatic view of an averaging oxygen sensing probe/analyzer in accordance with another embodiment of the present invention. Averaging conduit 400 is similar to conduit 300 (shown in FIG. 5) and like components are numbered similarly. Averaging conduit 400 is provided with an end scoop 402 positioned at a distal end of conduit 400. End scoop 402 is configured to capture a portion of flue gas proceeding along the direction indicated by arrow 404 and direct that flue gas axially within pipe 400 toward distal end 306. In this way, flow from the various inlet apertures or nozzles 304 to distal end 306 is facilitated by the additional flow created by end scoop 402.

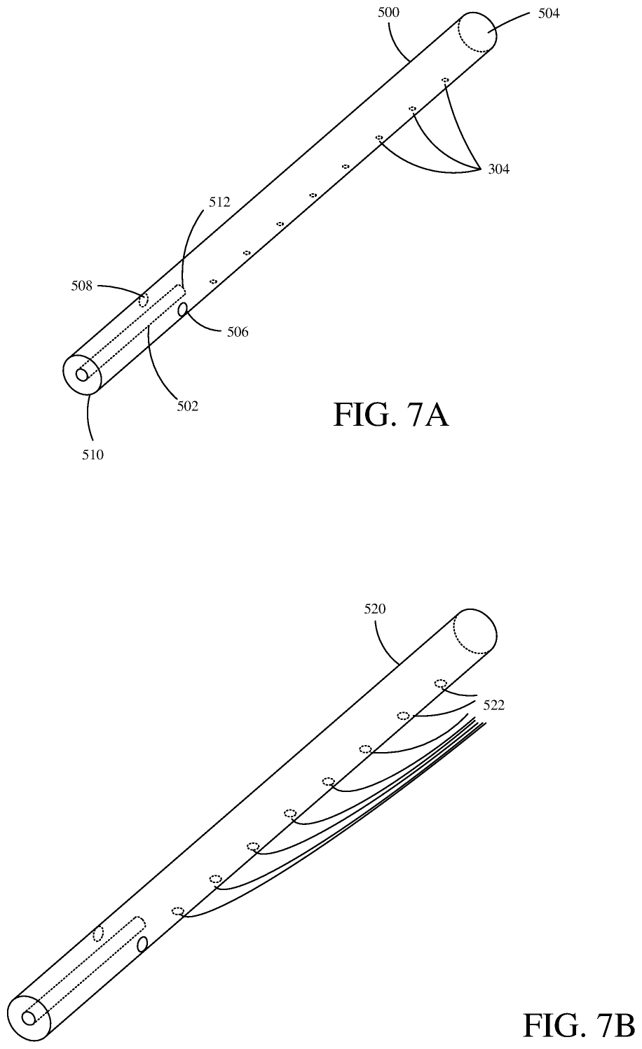

FIGS. 7A and 7B are diagrammatic perspective views of averaging conduits for an in-situ oxygen probe/analyzer in accordance with embodiments of the present invention. As shown in FIG. 7A, conduit 500 includes a plurality of evenly spaced inlet nozzles or apertures 304 that extend from probe receiving portion 502 to distal end 504. Additionally, unlike the previous embodiments described above, averaging conduit 500 includes a plurality of exit apertures or nozzles 506, 508. As can be seen, apertures 506, 508 are closer to proximal end 510 of conduit 500 than end 512 of probe receiving portion 502. Accordingly, the sensor or sensing region of the probe within probe receiving portion 502 will be positioned further from proximal end 510 than exit apertures 506, 508. Additionally, as can be seen in FIG. 7A, apertures 506, 508 are not disposed on the ultimate downstream surface of conduit 500, but instead are disposed approximately 90° from the inlet apertures. Additionally, the exit apertures 506, 508 are positioned diametrically opposite one another. Accordingly, the positioning and number of exit apertures can vary in accordance with embodiments of the present invention. Additionally, the size of inlet apertures 304 can be varied, as required.

FIG. 7B shows a perspective view of an averaging conduit 520 that is similar to averaging conduit 500 but employs a plurality of evenly-spaced larger inlet nozzles 522. Additionally, while FIGS. 7A and 7B show even spacing between the inlet nozzles or apertures, is also expressly contemplated that the spacing can be staggered or otherwise non-uniform in any suitable manner.

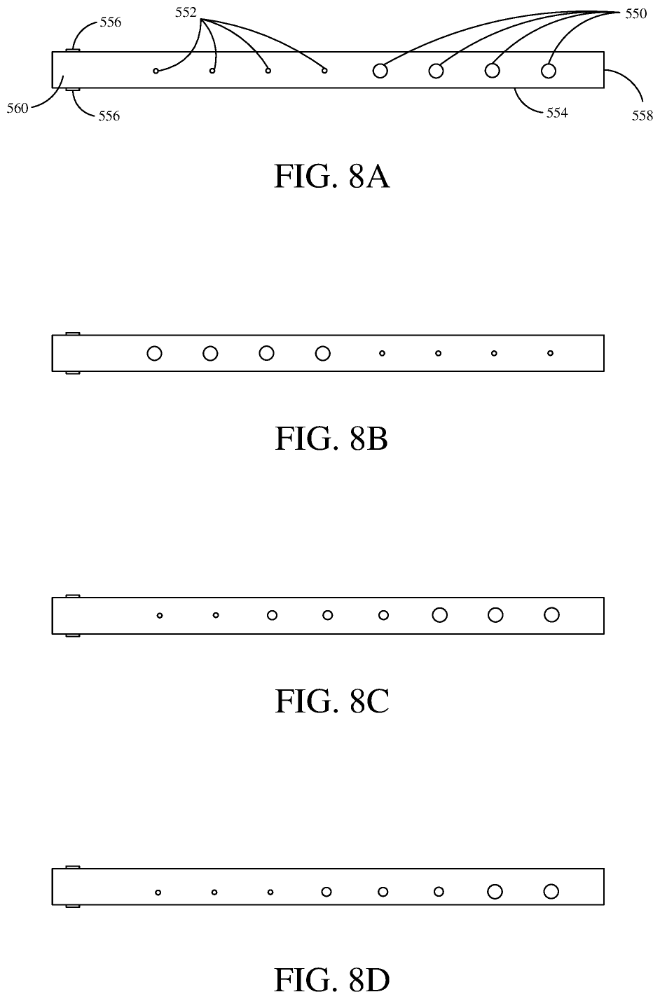

FIGS. 8A-8D show various embodiments of averaging conduits where the diameters of the inlet apertures vary on each averaging conduit. For example, FIG. 8A shows 8 such inlet apertures or nozzles 550, 552 on averaging conduit 554. Additionally, FIG. 8A shows averaging conduit 554 having a plurality of exit apertures 556 disposed approximately 90° from the inlet apertures and diametrically opposite one another. As can be seen in FIG. 8A, inlet apertures or nozzles 550 closer to distal end 558 have a larger diameter than inlet apertures 552, which are closer to proximal end 560 of conduit 554. Of course, if required, the situation may be reversed, such as shown in FIG. 8B. Further, three or more different diameters of inlet apertures can be used as shown in FIGS. 8C and 8D.

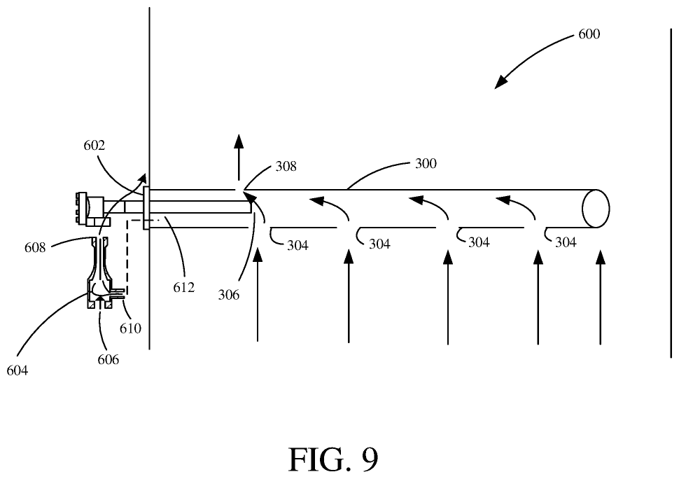

FIG. 9 is a diagrammatic view of a in-situ oxygen probe/analyzer in accordance with an embodiment of the present invention. System 600 bears some similarities to that described with respect to FIG. 5, and like components are numbered similarly. However, system 600 includes an active suction device that is mounted on or otherwise coupled to flange 602 in order to actively draw flue gas from inlets 304 past distal end 306. In the illustrated embodiment, the active suction device is an eductor 604 having an inlet 606, and an output 608. A suction port 610 is fluidically coupled to the interior 612 of conduit 300 such that when eductor 604 operates, suction is created at the proximal end of conduit 300 to draw the flue gas toward distal end 306. The output of eductor 604 may then be returned to the duct. Eductor 604 is merely one example of an active device to create flow within averaging pipe conduit.

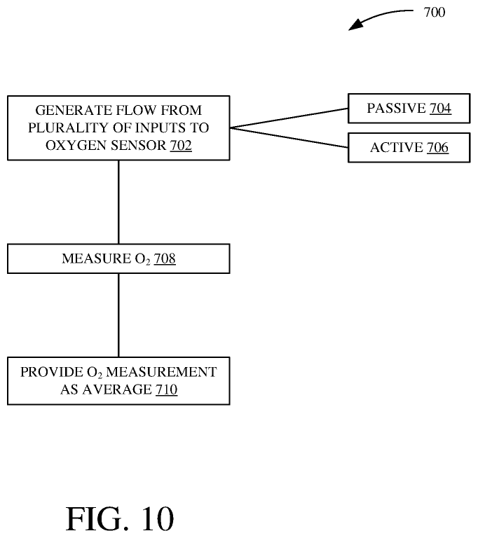

FIG. 10 is a flow diagram of a method of providing an average oxygen concentration of stratified flue gas using a single oxygen probe/analyzer in accordance with an embodiment of the present invention. Method 700 begins at block 702 where flow is a generated from a plurality of inputs that receive flue gas from at least two different positions within the flue toward a single oxygen sensor, such as sensor 112. The flow can be passive, as indicated at block 704, or active, as indicated at block 706. An example of an active flow includes the utilization of an eductor, as described with respect to FIG. 9.

Next, at block 708, the system measures the oxygen concentration using a single zirconia-based sensor, such as cell 112 (shown in FIG. 2). The response of this sensor is indicative of the oxygen concentration contacting the sensor cell. Since the flow of flue gas is from a plurality of inputs, the sensor response is a physical combination of the two inputs and may roughly be considered to be an average of the inputs. Accordingly, at block 710, the controller or electronics of the transmitter provides the measured oxygen concentration parameter as an average oxygen concentration for the flue gas as an output. This output may be provided as a local displayed output, and/or may be communicated over a process communication network, such as FOUNDATION™ Fieldbus, or WirelessHART (IEC62591). In any event, a single process intrusion (mounting of the probe through the flue/duct wall) can generate a reliable and effective average oxygen concentration value for the entire flue.

Although the present invention has been described with reference to preferred embodiments, workers skilled in the art will recognize that changes may be made in form and detail without departing from the spirit and scope of the invention.

What is claimed is:

1. An in-situ averaging combustion analyzer comprising:
a housing;
a probe coupled to the housing at a proximal end and having a distal end configured to extend into a flue, the probe containing an oxygen sensing cell proximate the distal end;
electronics disposed in the housing and coupled to the zirconia-based oxygen sensing cell, the electronics being configured to measure an electrical characteristic of the oxygen sensing cell and calculate an oxygen concentration value;
an averaging conduit disposed about the probe, the averaging conduit having a plurality of inlets spaced at different distances from the distal end of the probe, the averaging conduit having at least one outlet positioned between the distal end and the proximal end of the probe; and
wherein the electronics is configured to provide an average oxygen concentration output based on the calculated oxygen concentration value.

2. The in-situ averaging combustion analyzer of claim 1, wherein the oxygen sensing cell is a zirconia-based oxygen sensing cell.

3. The in-situ averaging combustion analyzer of claim 2, wherein the plurality of inlets is disposed on a downstream side of the averaging conduit.

4. The in-situ averaging combustion analyzer of claim 2, wherein the plurality of inlets have different diameters.

5. The in-situ averaging combustion analyzer of claim 2, wherein the plurality of inlets comprise a first plurality of inlets having a first diameter and a second plurality of inlets having a second diameter that is smaller than the first diameters.

6. The in-situ averaging combustion analyzer of claim 5, wherein the first plurality of inlets is disposed nearer proximal end of the probe than the second plurality of inlets.

7. The in-situ averaging combustion analyzer of claim 5, wherein the second plurality of inlets is disposed nearer the proximal end of the probe than the first plurality of inlets.

8. The in-situ averaging combustion analyzer of claim 2, wherein the plurality of inlets comprise at least three inlets spaced along the averaging conduit.

9. The in-situ averaging combustion analyzer of claim 8, wherein the at least three inlets are evenly spaced along the averaging conduit.

10. The in-situ averaging combustion analyzer of claim 2, wherein at least one outlet is disposed proximate a downstream side of the averaging conduit.

11. The in-situ averaging combustion analyzer of claim 2, wherein at least one outlet includes a plurality of outlets.

12. The in-situ averaging combustion analyzer of claim 11, wherein the plurality of outlets are disposed diametrically opposite from one another.

13. The in-situ averaging combustion analyzer of claim 11, wherein the plurality of outlets are disposed approximately 90 degrees from at least two inlets.

14. The in-situ averaging combustion analyzer of claim 2, and further comprising a flow assist device configured to increase flue gas flow from at least two inlets to the zirconia-based oxygen sensing cell.

15. The in-situ averaging combustion analyzer of claim 14, wherein the flow assist device is an end scoop mounted to a distal end of the averaging conduit.

16. The in-situ averaging combustion analyzer of claim 14, wherein the flow assist device is an eductor mounted proximate the housing.

17. The in-situ averaging combustion analyzer of claim 2, wherein the averaging conduit is a pipe.

18. An averaging conduit for a zirconia-based combustion analyzer, the averaging conduit comprising:
a mounting portion configured to be mounted relative to a combustion probe, the mounting portion comprising a proximal end;
a sidewall extending from the mounting portion to a distal end, the sidewall having a downstream surface and an upstream surface, the sidewall being configured to receive a probe of a combustion analyzer;
a plurality of inlet apertures positioned proximate the upstream surface; and
at least one exit aperture positioned proximate the downstream surface, the at least one exit aperture being disposed closer to the proximal end than the plurality of inlet aperture.

19. The averaging conduit of claim 18, wherein the mounting portion is configured to mount to the combustion probe.

20. The averaging conduit of claim 18, wherein the mounting portion is configured to mount to a flange of the combustion analyzer.

21. The averaging conduit of claim 18, wherein the averaging conduit is a pipe.

22. A method of providing an average oxygen value relative to stratified flue gas in a duct, the method comprising:
generating flow from a plurality of inputs to a single zirconia-based oxygen sensor; measuring a response of the single zirconia-based oxygen sensor; and
providing an average oxygen concentration value output based on the measured response of the single zirconia-based oxygen sensor.
